(19) United States
(12) Patent Application Publication
(10) Pub. No.: US 2020/0013493 A1
(43) Pub. Date: Jan. 9, 2020

(54) Consumer-Oriented Biometrics Data Management and Analysis System for Personalized Analysis, Insights, and Predictive Blood Glucose Response

(71) Applicant: ProductVisionaries, LLC(DBA BasilHealth), Los Altos, CA (US)

(72) Inventor: Kate Moloney-Egnatios, Los Altos, CA (US)

(21) Appl. No.: 16/576,043

(22) Filed: Sep. 19, 2019

Related U.S. Application Data
(63) Continuation-in-part of application No. 14/273,266, filed on May 8, 2014, now Pat. No. 10,453,562.

(51) Int. Cl. G16H 10/60; G16H 10/40; G16H 50/30; G16H 50/20; G16H 10/20

(52) U.S. Cl. CPC G16H 10/60; G16H 10/40; G16H 80/00; G16H 50/20; G16H 10/20; G16H 50/30

(57) ABSTRACT

An embodiment may involve receiving a set of information including data points for health-related data for a user. The embodiment may also involve performing, by an analytics engine, tests between (i) a series of one or more blood sugar levels from the user measured at points in time, and (ii) data representing each of a normal blood sugar response, a pre-diabetic blood sugar response, and a diabetic blood sugar response. The embodiment may also involve based on the tests, making a conclusion, by the analytics engine, that the series of one or more blood sugar levels from the user indicates the normal blood sugar response, the pre-diabetic blood sugar response, or the diabetic blood sugar response. The embodiment may also involve adding an indication of the conclusion to a comprehensive health profile for the user.

### CROSS-REFERENCE TO RELATED APPLICATION

This application is a continuation-in-part of and claims priority to U.S. patent application Ser. No. 14/273,266, filed May 8, 2014, which is hereby incorporated by reference in its entirety.

### BACKGROUND

Direct to consumer health care is a growing market with revenues that can be measured in the hundreds of billions of dollars. A focus on consumer health care requires specialized skill set and real innovation concentrated and applied at the intersection of consumer health, medical, and lifestyle data. Now, more than ever, individuals demand access to their own health and medical information, and the privacy of and control over their health and medical data.

For example, in the traditional healthcare and health provider model, an individual manages their health records in paper folders which are fetched by each individual health provider on a one-off basis and requires time, money, and high effort in order to obtain one single record of one single visit. Even further, the traditional healthcare model requires the individual visit a health care professional such as a doctor, to request medical laboratory tests. The healthcare professional or doctor may order medical laboratory test(s) on behalf of the consumer, and the individual often must take the time to visit a separate medical laboratory for a blood draw for these tests. The health care provider then receives the lab result(s) in typically anywhere from two to six weeks on behalf of the individual. The individual is requested to make yet another visit to the health care provider to get the results, or a waits a phone call from the health care provider (some estimates indicate that in 30% of cases the health care provider never calls).

In some cases, the results may never make it back to the individual at all due to health care professional or the medical laboratory error, administration error, negligence, or simple human error. The individual also may fail to follow-up with the health care provider if they do not ever hear from the health care provider. Alternatively, if the individual and health care provider do in fact successfully connect, the raw test results and any interpretation of these results would be mediated by the health care provider, rather than being under the control of the individual. Yet further, these results might not be viewed in the context of the individual's overall health.

### SUMMARY

Consumers or individuals may obtain more control over management of their personal health through the use of an electronic consumer-oriented biometrics information system for quantitative and qualitative personal health and medical data. This system, which may include one or more computing devices arranged to communicate over the Internet and/or other networks, may store quantitative and qualitative consumer biometrics and health information regarding one or more individual users. This information and data may include automatically or manually collected personal information (e.g., name, age, userid, password, personal identification number, or PIN, code), biographical information (e.g., gender, age, race, family medical history), current or historical biometric information collected by the user, the medical industry, mobile apps, trackers, medical devices or personal devices such as a watch, or questionnaires (e.g., EKG, ECG, brain waves and neural activity, fingerprint, retina scan, eye tracking, pulse, heart rate, sleep, stress information, fitness data, tracker device data, app data, medical device data, medical laboratory data, bodily fluid data, urine, stool, historical health or activity data), pharmaceutical medicines and/or other drugs, vitamins or supplements used by the individual or users, current or historical medical laboratory tests ordered by the individual or user, and any or all medical laboratory test results for the users, nutrition and diet information, self-reported health or medical conditions by the individual or user(s), food/environmental/medications/applied material and/or textile allergy information or chemical sensitivity data, camera-driven health data, air quality data, environmental pollution data, mobile activity, history and usage data, genetic DNA, venous measurements, audio data, sound data, skin health data, feet imprint data, cheek cell swab data, nasal bacterial, virus and tissue data, facial temperature data, pregnancy and birth status data and history, fertility, stool sample data, urine chemistry data, auditory hearing data, saliva data, breathalyzer data, body temperature and thermal activity, voice, facial recognition, breath chemistry, electromagnetic data and/or radio frequency data, fitness, exercise, heavy metals or other chemistry data, alcohol consumption, toxicity data, biohazard data, sensor data, sexual health status, activity and/or sexual health disease data, and other qualitative health data including but not limited to personal mood, stress levels, health conditions, medical conditions, personal journal data and history, work health hours and stress levels, relationship status data and history, emotional status data and history, and personal health information regarding the individual or users' answers to various health questionnaires, macro-level population health data as well as other types of qualitative and quantitative consumer-oriented biometrics health-related data.

With these quantitative and qualitative sources and various types of information all in one centralized location, an individual or user may be able to better review, track, organize and manage his or her own health care, and get more personalized insights based on predictive analysis rather than rely on various doctors, hospitals, laboratories, pharmacies, government data, and/or web sites, and so on that are currently fragmented, disconnected, and may not ever effectively communicate with one another. Further, with the advent of wearable health-tracking devices, such as digital pedometers, heart-rate monitors, fitness monitors, fertility monitors, blood sugar monitors, watches, mobile apps, medical devices, other trackers and so on, the individual or user may be able to automatically measure a wealth of personalized information and data regarding or affecting their human body, and create a 360-degree picture of their health based on a set of qualitative and quantitative consumer-oriented biometric data. Thus, users may be able to see how they're doing, better organize, review, track and manage the information, and ultimately make better decisions about their everyday health. The data and measurements may also be integrated into the health information system.

For the first time, with easy access to a broad range of personalized consumer biometrics data, the consumer-oriented data management health information and management system may be able to perform personalized analyses and predictions on the data to create a personalized 360-degree health profile, discover correlations and trend-lines that might not be otherwise apparent, or even detect adverse interactions or health/medical condition status or precursor(s). Further, the consumer-oriented biometric health management and analysis information system may also be able to serve as a unique ecommerce portal by which individuals or users can access a new set of innovative tools by which to better manage their own health. For example, an individual may search for, browse, select and order their own medical laboratory test(s) kits online, and from directly inside the consumer-oriented biometric health management and analysis information system. Lab results from these medical laboratory tests may be securely integrated to, or from, the medical laboratory, delivered directly into the user's data and account in the consumer-oriented biometric health management and analysis information system.

Similarly in examples, an individual or user may upload, select, review, authorize, fill/re-fill/order their pharmacy prescriptions online, and from directly inside the consumer-oriented biometric health management and analysis information system. The prescription (RX) and data may be securely integrated to, or from, the pharmacy, delivered directly into the user's data and account in the consumer-oriented biometric health management and analysis information system.

Moreover, the consumer-oriented biometric health management and analysis information system may also fetch, collect, and aggregate relevant scientific research, environmental, lifestyle, health or medical-related information or content that may be of interest to the user based on their unique biometric data. Sources may include medical journals, scientific research, medical trials, pharmaceutical research or information, articles, papers, links to web sites, data sources, and so on. As a result, the consumer oriented biometrics health management and analysis information system may be tailored to the specific needs and profile of the individual, user, and/or consumers in general, and not dependent on a health care professional, physician, hospital, mobile app, or particular access to medical devices, information upload devices, health tracking and monitoring devices, medical/health/lifestyle professionals, health or medical laboratory tests, medical laboratories or pharmacies.

Accordingly, in a first example embodiment, a set of information including manually-entered health-related data for a user, automatically collected health-related data for the user, and test results for the user may be received. In response to receiving the set of information, the manually entered health-related data for the user, the automatically collected health-related data for the user, and the test results for the user may be integrated into a comprehensive health profile for the user. Upon a request made on behalf of the user, at least part of the comprehensive health profile may be provided to the user.

A second example embodiment may involve receiving, by a server device, a set of information including pluralities of data points for health-related data for a user. Possibly in response to receiving the set of information, an analytics engine associated with the server device may perform tests between (i) a series of one or more blood sugar levels from the user, and (ii) data representing each of a normal blood sugar response, a pre-diabetic blood sugar response, and a diabetic blood sugar response, wherein the series of one or more blood sugar levels from the user is part of the health-related data for the user. Possibly based on the tests, the analytics engine may make a conclusion that the series of one or more blood sugar levels from the user indicates the normal blood sugar response, the pre-diabetic blood sugar response, or the diabetic blood sugar response. The server device may add an indication of the conclusion to a comprehensive health profile for the user. Upon a request made on behalf of the user, the server device may provide at least part of the comprehensive health profile, including the conclusion.

A third example embodiment may include a non-transitory, computer-readable storage medium, having stored thereon program instructions that, upon execution by a computing device, cause the computing device to perform operations in accordance with the first and/or second example embodiment.

A fourth example embodiment may include a computing device containing at least a processor and data storage. The data storage may include program instructions that, when executed by the processor, cause the computing device to perform operations in accordance with the first and/or second example embodiment.

These as well as other embodiments, aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 is a high-level depiction of a health information system, according to an example embodiment.

FIG. 2 illustrates a schematic drawing of a computing device, according to an example embodiment.

FIG. 3 illustrates a schematic drawing of a networked server cluster, according to an example embodiment.

FIG. 4A is an information flow diagram, according to an example embodiment.

FIG. 4B is an information flow diagram, according to an example embodiment.

FIG. 5 is an information flow diagram, according to an example embodiment.

FIG. 6 is a correlational analysis chart, according to an example embodiment.

FIG. 7 is a longitudinal trend analysis chart, according to an example embodiment.

FIG. 8 is a flow chart, according to an example embodiment.

FIG. 9 is another flow chart, according to an example embodiment.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Other embodiments can be utilized, and other changes can be made, without departing from the scope of the subject matter presented herein.

Thus, the example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

### 1. OVERVIEW

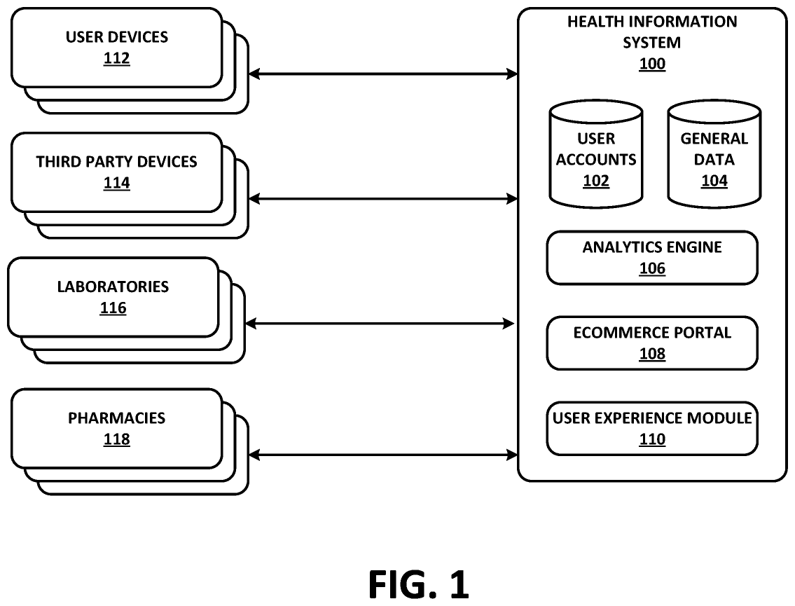

FIG. 1 is a high-level depiction of a health information system 100 and its connectivity to other entities. Health information system 100 may be software that operates on one or more computing devices, such as client and/or server devices. In some embodiments, health information system 100 may be implemented as a “cloud-based” service on Internet servers, accessible via web pages, for example. In other embodiments, health information system 100 may consist of client and/or server software.

Regardless, health information system 100 may contain or have access to user accounts 102, general data 104, analytics engine 106, ecommerce portal 108, and user experience module 110.

User accounts 102 may be a database and/or some other organization of information that represents attributes of one or more users of health information system 100. Thus, for each user, user accounts 102 may include a username, email address, physical address, phone number, and/or billing information. User accounts 102 may also include representations of health information (such as test results, questionnaire results, and quantitative, biometric data related to user health) and ecommerce transactions (such as test kits ordered) for each user. Users may be able to tag or mark some aspects of their profile as “public” or “semi-private” data that can be shared with a medical professional, family, and/or friends. By default, some or all of a user's data may be marked as “private” until the user marks the data otherwise.

General data 104 may include health-related information that is not specific to a particular user. Thus, general data may include height and weight charts, nutrition and diet information, exercise information, health-related articles, and so on. Users of health information system 100 may access general data 104 at their leisure or when they have a specific health-related question. General data may be browseable and/or searchable.

Analytics engine 106 may be software arranged to calculate correlations between the data associated with a user in user accounts 102. For instance, analytics engine 106 may be able to determine relationships between a user's diet, medications and his or her reported mood. Further, analytics engine 106 may be able to track longitudinal trends related to a user's health. As an example, analytics engine 106 might find a long term trend between a user's weight and one or more of his or her diet, sleep, blood sugar, heart rate, hydration levels, and so on. Analytics engine may also be able to calculate these correlations and longitudinal trends across multiple users.

Ecommerce portal 108 may be an online store that allows users to shop for health-related products and services. For instance, ecommerce portal may provide over-the-counter medicines, health-related books, exercise equipment, vitamins and supplements, specialty food products, specialty health and medical products, mobile application downloads, pet health and pet health products, health tracking and monitoring devices, electronic gadgets, over-the-counter health products, and self-testing products and home test kits.

As an example, for the test kits, the user may order a kit, and have it shipped to the user. The user may perform the test and ship the results to an associate laboratory. After completing the testing procedure, the user may use ecommerce portal 108 to find an appropriate laboratory that can provide test results, or the user may determine a laboratory in some other fashion. The user would ship a sample associated with the test (e.g., a blood sample, saliva sample, skin sample, etc.) to one of the laboratories. The selected laboratory would then receive the sample, perform testing on the sample, and upload and/or post the results to health information system 100, and these results may become part of the user's account in user accounts 102.

User experience module 110 may provide the “front end” or user interface to health information system 100. Thus, user experience module 100 may be arranged to provide users intuitive access to information in their accounts, as well as to general data 104. For instance, a particular user might be able to customize their user experience so that they are provided with more information that they are interested in, and less information of general interest.

Health information system 100 may facilitate online access from one or more user devices 112, third party devices 114, and/or laboratories 116. Each of user devices 112, third party devices 114, and laboratories 116 may be a computing device, and these computing devices may be interconnected by a computer network, such as the Internet or one or more private networks.

Each of user devices 112 may correspond to a human user of health information system 100. Such a user device may be a mobile device, laptop, tablet, PC, etc., that the user utilizes to access health information system 100. Alternatively, some of these user devices may be wearable computing devices, health tracking devices, and/or self-monitoring devices (e.g., digital pedometers, heart rate monitors, blood sugar monitors, etc.) that are configured to upload or post gathered health data to the user's account.

Each of third party devices 114 may correspond to a human or automated entity that is permitted to have at least limited access to some aspects of user accounts 102. For instance, third party devices 114 may be associated with medical professionals who are granted access to test results or other information in one or more of user accounts 102. In some situations, third party devices 114 may include doctors, doctor's assistants, hospitals, clinics, and so on, and/or devices or software (e.g., mobile devices, laptops, tablets, PCs, applications, etc.) used by these individuals or organizations.

Laboratories 116 may correspond to one or more medical testing laboratories that may be able to, with a user's permission, upload test results to the user's account (e.g., update or post the results to health information system 100 via an appropriate application programming interface (API)). However, other arrangements are possible, such as two or more human users sharing the same user device, two or more medical professionals sharing the same third party device, and so on.

Pharmacies 118 may correspond to one or more online or physical entities that can fulfill prescriptions for medicines and/or drugs. Users may communicate with pharmacies 118 either directly or through health information system 100.

Health information system 100, as well as any other device or function associated with the architecture of FIG. 1, can represent, be operated on, or be operated by one or more computing devices. These computing devices may be organized in a standalone fashion, in networked computing environments, or in other arrangements. Examples are provided in the next section.

### 2. EXAMPLE COMPUTING DEVICES AND CLOUD-BASED COMPUTING ENVIRONMENTS

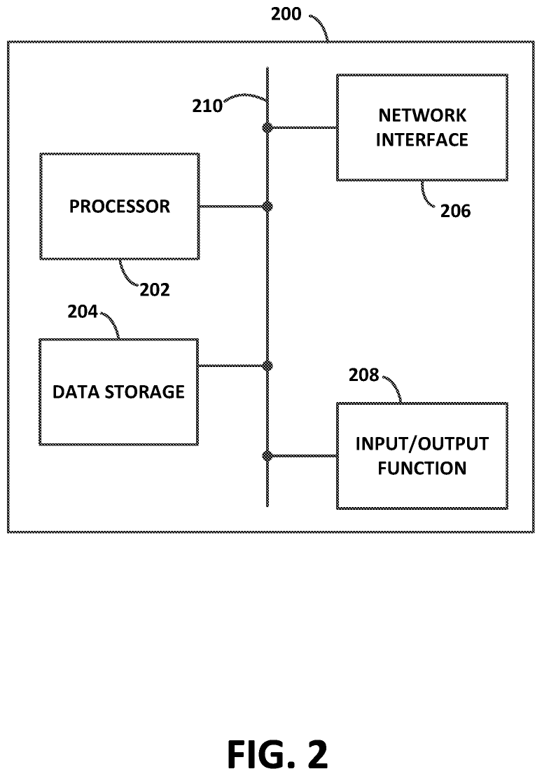

FIG. 2 is a simplified block diagram exemplifying a computing device 200, illustrating some of the functional components that could be included in a computing device arranged to operate in accordance with the embodiments herein. Example computing device 200 could be a personal computer (PC), laptop, server, or some other type of computational platform. For purposes of simplicity, this specification may equate computing device 200 to a server from time to time. Nonetheless, it should be understood that the description of computing device 200 could apply to any component used for the purposes described herein.

In this example, computing device 200 includes a processor 202, a data storage 204, a network interface 206, and an input/output function 208, all of which may be coupled by a system bus 210 or a similar mechanism. Processor 202 can include one or more central processing units (CPUs), such as one or more general purpose processors and/or one or more dedicated processors (e.g., application specific integrated circuits (ASICs), digital signal processors (DSPs), network processors, etc.).

Data storage 204, in turn, may comprise volatile and/or non-volatile data storage and can be integrated in whole or in part with processor 202. Data storage 204 can hold program instructions, executable by processor 202, and data that may be manipulated by these instructions to carry out the various methods, processes, or functions described herein. Alternatively, these methods, processes, or functions can be defined by hardware, firmware, and/or any combination of hardware, firmware and software. By way of example, the data in data storage 204 may contain program instructions, perhaps stored on a non-transitory, computer-readable medium, executable by processor 202 to carry out any of the methods, processes, or functions disclosed in this specification or the accompanying drawings.

Network interface 206 may take the form of a wireline connection, such as an Ethernet, Token Ring, or T-carrier connection. Network interface 206 may also take the form of a wireless connection, such as IEEE 802.11 (Wifi), BLUETOOTH®, or a wide-area wireless connection. However, other forms of physical layer connections and other types of standard or proprietary communication protocols may be used over network interface 206. Furthermore, network interface 206 may comprise multiple physical interfaces.

Input/output function 208 may facilitate user interaction with example computing device 200. Input/output function 208 may comprise multiple types of input devices, such as a keyboard, a mouse, a touch screen, and so on. Similarly, input/output function 208 may comprise multiple types of output devices, such as a screen, monitor, printer, or one or more light emitting diodes (LEDs). Additionally or alternatively, example computing device 200 may support remote access from another device, via network interface 206 or via another interface (not shown), such as a universal serial bus (USB) or high-definition multimedia interface (HDMI) port.

In some embodiments, one or more computing devices may be deployed in a networked architecture. The exact physical location, connectivity, and configuration of the computing devices may be unknown and/or unimportant to client devices. Accordingly, the computing devices may be referred to as “cloud-based” devices that may be housed at various remote locations.

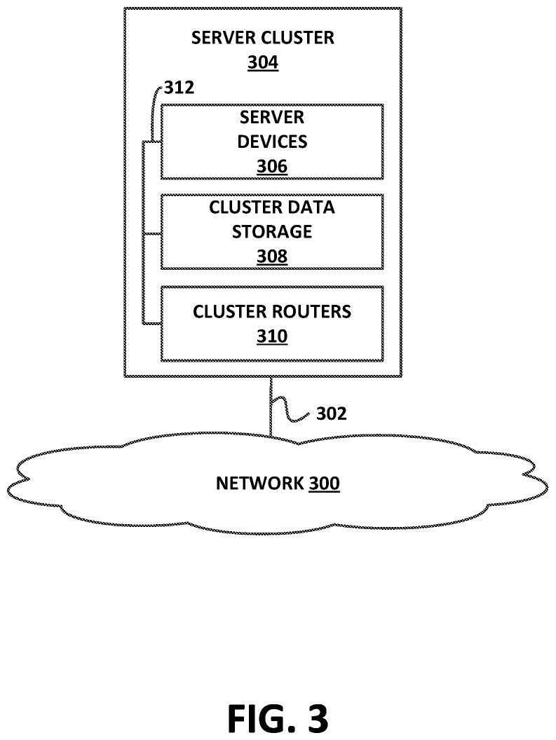

FIG. 3 depicts a cloud-based server cluster 304 in accordance with an example embodiment. In FIG. 3, functions of computing device 200 may be distributed between server devices 306, cluster data storage 308, and cluster routers 310, all of which may be connected by local cluster network 312. The number of server devices, cluster data storages, and cluster routers in server cluster 304 may depend on the computing task(s) and/or applications assigned to server cluster 304.

For example, server devices 306 can be configured to perform various computing tasks of computing device 200. Thus, computing tasks can be distributed among one or more of server devices 306. To the extent that these computing tasks can be performed in parallel, such a distribution of tasks may reduce the total time to complete these tasks and return a result.

Cluster data storage 308 may be data storage arrays that include disk array controllers configured to manage read and write access to groups of hard disk drives. The disk array controllers, alone or in conjunction with server devices 306, may also be configured to manage backup or redundant copies of the data stored in cluster data storage 308 to protect against disk drive failures or other types of failures that prevent one or more of server devices 306 from accessing units of cluster data storage 308.

Cluster routers 310 may include networking equipment configured to provide internal and external communications for the server clusters. For example, cluster routers 310 may include one or more packet-switching and/or routing devices configured to provide (i) network communications between server devices 306 and cluster data storage 308 via cluster network 312, and/or (ii) network communications between the server cluster 304 and other devices via communication link 302 to network 300.

Additionally, the configuration of cluster routers 310 can be based at least in part on the data communication requirements of server devices 306 and cluster data storage 308, the latency and throughput of the local cluster networks 312, the latency, throughput, and cost of communication link 302, and/or other factors that may contribute to the cost, speed, fault-tolerance, resiliency, efficiency and/or other design goals of the system architecture.

### 3. EXAMPLE INFORMATION FLOWS

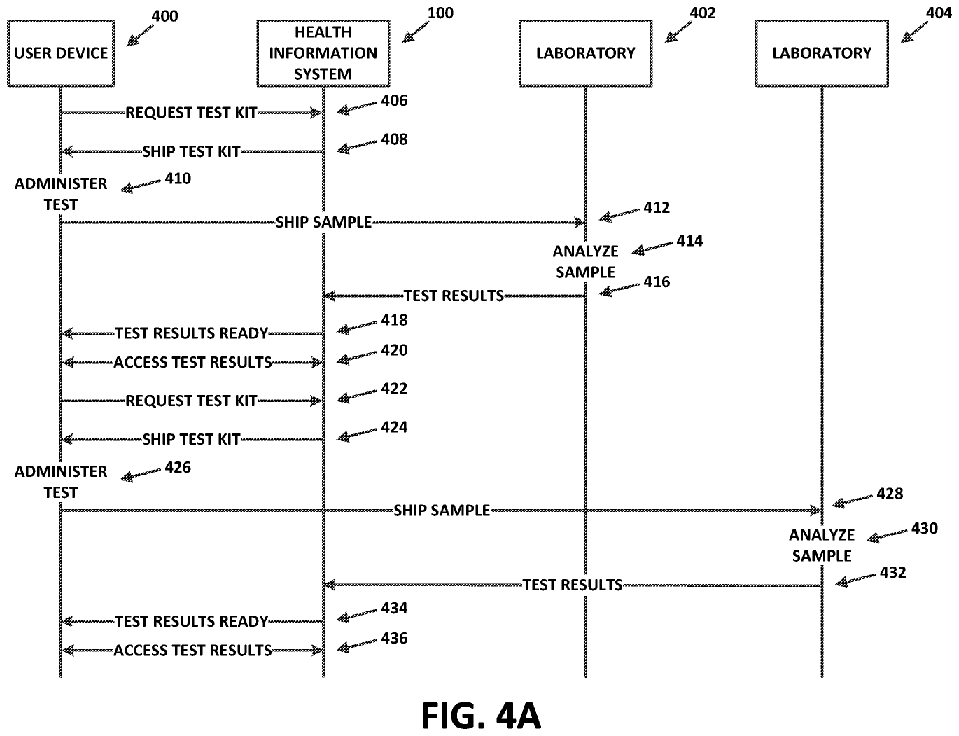

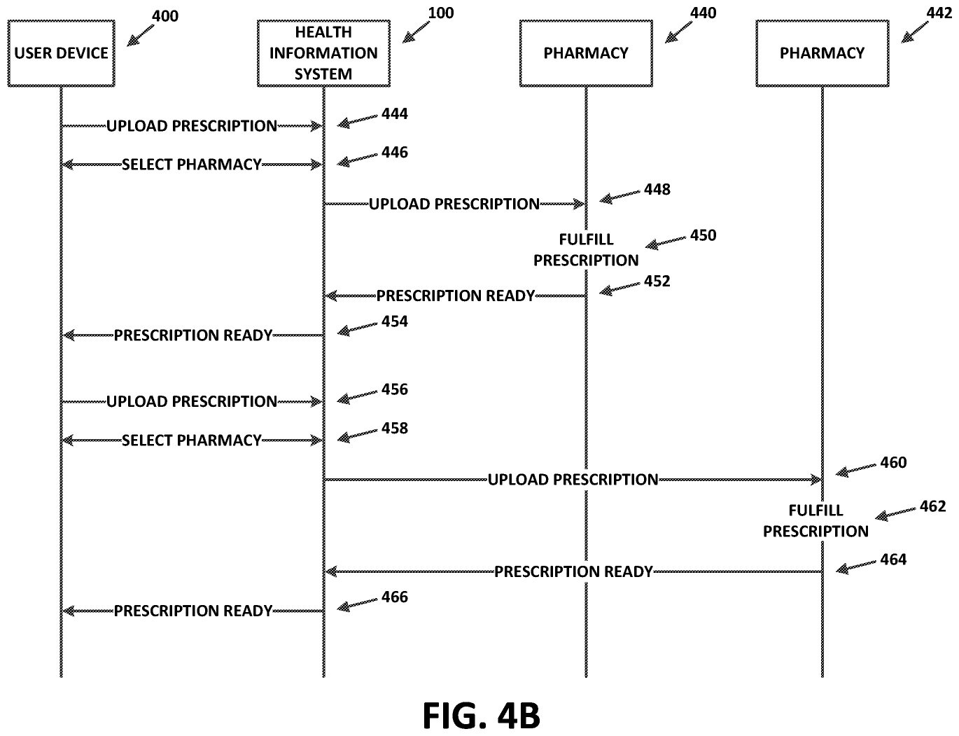

FIG. 4A is an example ecommerce information flow that may be supported by health information system 100. Health information system 100 may provide an online ecommerce portal at which various types of home health test kits can be ordered. User device 400, one of user devices 112, may be in communication with health information system 100. Laboratory 402 and laboratory 404 may also be in communication with health information system 100. These laboratories may be arranged to analyze test samples (e.g., blood samples, saliva samples, skin samples, etc.). Each of these entities may communicate with one another via a computer network such as the Internet.

In traditional health care, a patient (e.g., user) typically attends an appointment with a medical professional (e.g., a doctor) in order to obtain a health test (e.g., a blood sample, saliva sample, skin samples, etc.). The medical professional may either collect a sample from the patient or send the patient to a laboratory for sample collection. Once the sample is collected, the laboratory (which may have been selected by the medical professional), analyzes the sample and sends the test results to the medical professional. The user obtains information regarding the test results from the medical professional, and may not be granted access to the raw test results.

Some users may prefer to have more privacy and control over their health care and medical information. Instead of, or in addition to visiting a medical professional, these users may order home health test kits. The test kits may be provided by health information system 100 directly, or by an entity associated with the operation of health information system 100 or a laboratory.

The users may also be able choose which of several possible laboratories to send the sample collected by these kits, and the selected laboratories may have the capability to upload test results directly to health information system 100 (e.g., update or post the results to health information system 100 via an appropriate API). In this way, the user has access to, privacy over, and controls their own health information, and the entire procedure revolves around the user instead of the medical professional. Alternatively, the user may order a test kit that is associated with a particular laboratory, and then may send the sample to that laboratory.

Accordingly, at step 406, user device 400, on behalf of a user with an account on health information system 100, may transmit a request for a test kit to health information system 100. At step 408, health information system 100 may ship (e.g., via postal mail) the requested test kit to the user. In some embodiments, health information system 100 may forward the request for the test kit to a laboratory (e.g., laboratory 402 or laboratory 404), and the laboratory may ship the test kit directly to the user or via health information system 100.

At step 410, the user may administer the test, and at step 412, may ship a test sample (again, possibly by postal mail) to a laboratory, such as laboratory 402. At step 414, laboratory 402 may analyze the sample, and at step 416 may upload (or post via an API supported by health information system 100) the test results to health information system 100.

Possibly in response to receiving this upload, at step 418, health information system 100 may transmit a message to user device 400 that the test rests are ready. Then, at step 420, the user of user device 400 may access the test results.

In some situations, the user might want to use multiple laboratories for different tests. For instance, some laboratories may have a reputation for providing more accurate results for specific types of tests. Alternatively, the user might want to have two or more laboratories analyze the same types of samples so that the user can corroborate each laboratory's results. For instance, the user may draw two blood, two saliva, or two urine samples, and send each one to a different laboratory. Alternatively, the user may obtain one larger sample (e.g., blood, saliva, or urine), and send parts of that sample to different laboratories. If the results from the laboratories agree, the user can be reasonably confident that they are accurate. If the results do not agree, the user then knows that further testing is warranted.

Thus, at step 422, user device 400, on behalf of the user, may transmit a request for a test kit to health information system 100. At step 424, health information system 100 may ship the requested test kit to the user. The test kit may be the same type of test kit as was shipped in step 408, or may be a different type of test kit. Health information system 100 may forward the request for the test kit to a laboratory (e.g., laboratory 402 or laboratory 404), and the laboratory may ship the test kit directly to the user or via health information system 100.

At step 426, the user may administer the test, and at step 428, may ship a test sample (again, possibly by postal mail) to a laboratory, such as laboratory 404. At step 430, laboratory 404 may analyze the sample, and at step 432, may upload (or post via an API supported by health information system 100) the test results to health information system 100.

Possibly in response to receiving this upload, at step 434, health information system 100 may transmit a message to user device 400 that the test rests are ready. Then, at step 436, the user of user device 400 may access the test results.

The test results for either test may be raw test results, in that they may be original data from the laboratory with reference ranges and/or little to no interpretation. Possibly though analysis by health information system 100, the raw test results may be accompanied by an interpreted version of the test results, perhaps providing user-friendly explanations and/or highlights of the outcome of the test. User experience module 110 of health information system 100 may allow the user to view both the raw test results from the laboratory and user-friendly, interpreted results provided by health information system 100, perhaps with the ability to switch between views of the two.

Once test results are uploaded to health information system 100, the user may grant other parties access to at least some of this information. For instance, the user may grant access to the test results to medical professionals, family members, and/or friends.

In some embodiments, the user may first obtain an order for a test from a medical professional. The user may upload this order to his or her account, and then this information may be provided along with test kits requests 406 and 422.

FIG. 4B is another example ecommerce information flow that may be supported by health information system 100. Health information system 100 may serve as an intermediary between the user and one or more pharmacies. Thus, pharmacy 440 and pharmacy 442 may also be in communication with health information system 100. These pharmacies may online or physical stores that fulfill prescriptions.

At step 444, user device 400 may upload a prescription to health information system 100. The prescription might entail, for example, a doctor's order to provide the user with a particular medicine or drug. At step 446, via user device 400 and health information system 100, the user may select a pharmacy to fulfill the prescription. Health information system 100 may, for instance, recommend a particular pharmacy based on its location, the cost to fulfill the prescription, supported insurance plans, and/or user preference. Thus, health information system 100 may be arranged to automatically cross-reference or check prescription pricing, prescription ingredients, pharmacy location, and so on according to what is most important to the user, and then provide a list of one or more recommended pharmacies.

At step 448, health information system 100 may upload the prescription to a selected pharmacy, such as pharmacy 440. At step 450, pharmacy 440 may fulfill the prescription, and at step 452, pharmacy 440 may transmit an indication to health information system 100 that the prescription is ready. At step 454, health information system 100 may, in turn, transmit an indication to user device 400 that the prescription is ready (e.g., a push notification to the user's email or mobile device). The user may then choose to pick up the prescription in person or to have the prescription shipped to his or her location.

As was the case with laboratories, health information system 100 may support multiple pharmacies. For instance, the user might prefer to use a primary pharmacy when at home, but select a different one when he or she is travelling. Alternatively or additionally, different pharmacies may charge different amounts to fulfill the same prescription. Thus, the user might use one pharmacy to fulfill some prescriptions, but use another pharmacy to fulfill additional prescriptions.

Accordingly, at step 456, user device 400 may upload another prescription to health information system 100. At step 458, via user device 400 and health information system 100, the user may select a pharmacy to fulfill the prescription. At step 460, health information system 100 may upload the prescription to a selected pharmacy, such as pharmacy 442. At step 462, pharmacy 442 may fulfill the prescription, and at step 464, pharmacy 442 may transmit an indication to health information system 100 that the prescription is ready. At step 466, health information system 100 may, in turn, transmit an indication to user device 400 that the prescription is ready. The user may then choose to pick up the prescription in person or to have the prescription shipped to his or her location.

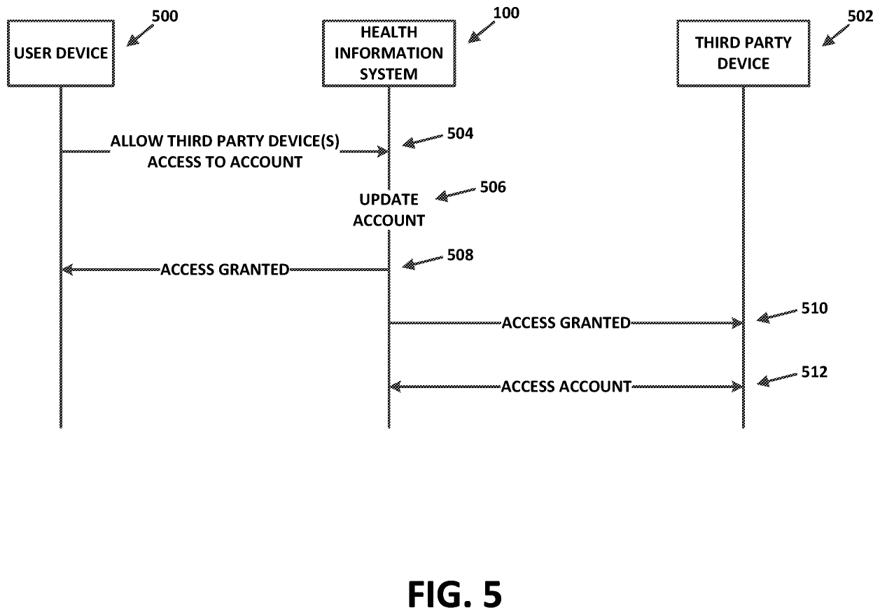

FIG. 5 is an example third party authorization information flow that may be supported by health information system 100. In FIG. 5, user device 500, one of user devices 112, may be in communication with health information system 100. Also, third party device 502, one of third party devices 114, may be in communication with health information system 100.

At step 504, user device 500 may allow one or more third party devices access to an account associated with user device 500. Thus, user device 500 may seek to grant third party device 502 access to the account.

At step 506, health information system 100 may update the account to indicate that third party device 502 and/or another account that is associated with third party device 502 is permitted to access parts of the account. At step 508, health information system 100 may transmit an indication to user device 500 that this access has been granted. Similarly, at step 510, health information system 100 may transmit an indication to third party device 502 that this access has been granted.

Then, at step 512, third party device 502 may access the account. This access may entail viewing and/or downloading test results stored in or available via the account.

Additionally, the user may be able to access a medical, health, and/or lifestyle expert via health information system 100. For instance as part of received test results, or after receiving the test results, the user may be presented with an option to review these results with an expert, or otherwise contact an expert. This contact may be via health information system 100, phone, text message, email, video call, etc.

Any of these embodiments may also be used for purposes of pet health as well. Thus, users may browse and order pet health test kits, obtain laboratory results, and share these results with veterinarians and other entities. In these cases, the information in user accounts 102 and general data 104 may include pet-related data.

### 4. EXAMPLE INFORMATION STORED IN A HEALTH INFORMATION SYSTEM

Possibly as part of an account of a particular user in user accounts 102, or part of general data 104, various types of data may be stored or accessible. This data may be in addition to test results, and may be divided into various categories, included or not limited to the following.

a. Manually Inputted Data

An account of user accounts 102 may include data related to a user's food, diet and nutrition, sleep, sexual activities, stress, fitness, exercise and activity, height, weight, hydration, blood sugar, blood pressure, other blood works, cholesterol, heart rate, respiratory rate, oxygen saturation, anger levels, female fertility and ovulation, female menstrual cycles, emotional and mental health, religion and spiritual health, social connections and social health, medical history, conditions and disease, doctors, hospitals and visit history, children's health, spouse health, baby health, and notes from friends, family, doctors, health practitioners.

b. Data from Monitoring Devices

An account of user accounts 102 may include data that can be collected via various types of health tracking and monitoring devices. These devices may include wearable computing devices, such as digital pedometers, heart rate monitors, and so on. Thus, data may be collected via wristbands, healthbands, watches, smartwatches, headbands, socks, shirts, fashion apparel, tricoders, home tracking devices, thermometers, diabetes and blood sugar monitoring devices, bandages, smartphones, smart wallets, children and baby monitors, children and baby fashion apparel, fashion accessories (e.g., bags, belts, pins, buttons, cuff links, scarves, etc.), textiles, eyewear, fashion, jewelry (necklaces, earrings, bracelets, etc.), bikes, electronic audio devices, cameras, sousveillance (data collection units worn by an individual), ear pieces, hearing aids, and so on.

c. General Data

Possibly as part of general data 104, health information system 100 may store or have access to various types of general information. This information may include, but is not limited to, health-related articles, academic papers, personal stories, advice from experts, links to other web sites, and so on.

### 5. EXAMPLE ANALYTICS

Analytics engine 106 may include various capabilities to analyze the data associated with an account, as well as general data 104, to draw conclusions from this information. Two example embodiments are provided below, one a correlation analysis and the other a longitudinal trend analysis. However, these embodiments are merely examples, and other embodiments may exist, and these embodiments may use correlational analysis, longitudinal trend analysis, a combination of both, or one or more additional techniques.

a. Correlations

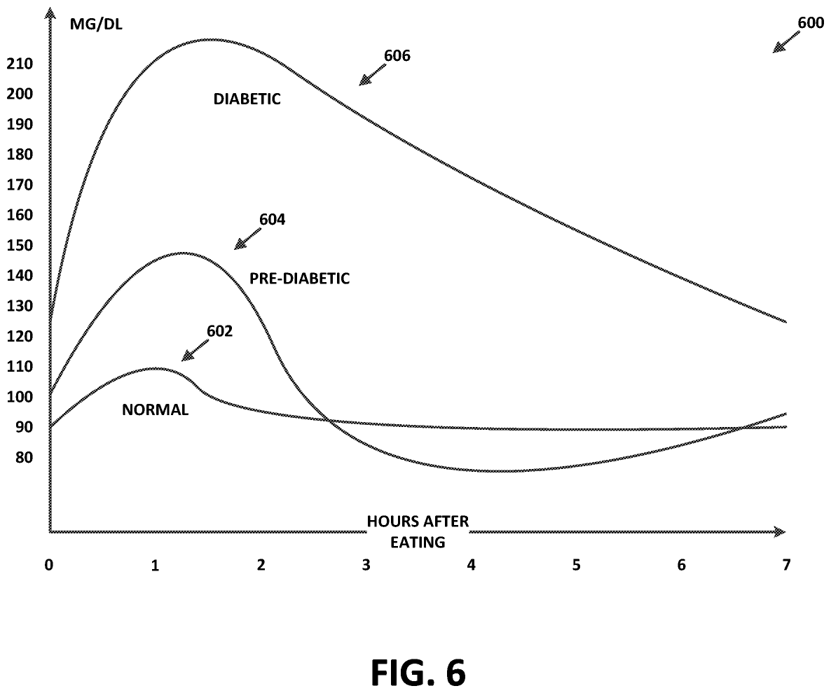

FIG. 6 illustrates example data that could be used in a correlation analysis.

Chart 600 is a graph that plots blood sugar levels versus the number of hours after eating a meal. Three example curves are plotted. Curve 602 indicates a normal blood sugar response, which starts at about 90 milligrams per deciliter (mg/dL) and peaks around 110 mg/dL one hour after eating, then fall back to 90 mg/dL. Curve 604 indicates a pre-diabetic blood sugar response, which starts at about 100 mg/dL, peaks around 150 mg/dL approximately 1.5 hours after eating, dips below 80 mg/dL for several hours after that, then returns to about 100 mg/dL. Curve 606 indicates a diabetic blood sugar response, which starts at about 125 mg/dL, peaks around 215 mg/dL approximately 2 hours after eating, and then returns to about 125 mg/dL.

Analytics engine 106 may use these curves to determine a likelihood of diabetes in a user. For instance, the user might be wearing a device that periodically measures the user's blood sugar levels. Alternatively, the user may manually test his or her blood sugar levels several times after eating. The time that the user ate the meal could also be manually or automatically collected.

Based on data points collected from the user, analytics engine 106 may compare the data points to curve 602, 604, and 606. For instance, analytics engine may conduct a regression analysis to determine a curve for the data points, and/or perform one or more goodness-of-fit tests between data collected from the user and these curves. Based on the outcome of these tests, analytics engine may conclude that the data points are more likely to indicate a normal response, pre-diabetic response, or a diabetic response, and the user may be informed accordingly. The goodness-of-fit test may be based on various statistical methods (e.g., Chi-Squared, Kolmogorov-Smirnov, sum of squares, or any form of regression analysis).

Alternative embodiments may involve the blood sugar tests being fasting blood sugar tests (e.g., taken while the user has not eating for some period of time, such as 8-14 hours). In other embodiments, the blood sugar tests may be within a few minutes (e.g., 0-10) of any one of the following time period: before eating, after eating, before going to bed at night, or after rising in the morning. Similar to FIG. 6, each of these time periods may be characterized by their own canonical blood sugar responses, represented as curves or data points covering the next several hours. In general, the data in such a representation can start at an approximate baseline, drop before eating, rise immediately after eating, peak approximately one hour after eating, then return or remain irregular within two hours after eating, before bed at night, and upon rising first thing in the morning.

b. Longitudinal Trends

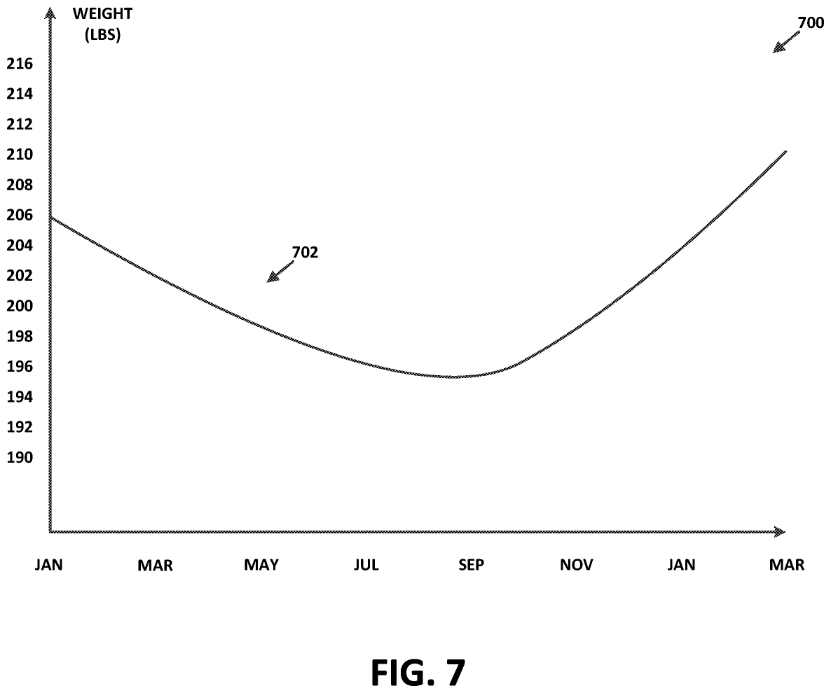

FIG. 7 illustrates example data that could be used in a longitudinal trend analysis. Chart 700 plots a male user's weight versus month over a period of 15 months. Curve 702 indicates that the user weighed about 206 pounds in January, and then lost weight steadily that year until about September. In September, the user's weight was about 195 pounds, but over the next six months, the user gained approximately 15 pounds until he weighed about 210 pounds in March of the next year.

Analytics engine 106 may obtain data regarding the user's weight at various points in time (e.g., once a day, one a week, twice a month, etc.) from, for instance, a digital scale or via manual entry. Analytics engine 106 may consider this data over the course of weeks, months, or years to determine the user's weight trends. The particular trend in FIG. 7 may indicate that the user gains weight in the winter months and loses it in the summer. Thus, analytics engine might recommend that the user more carefully plan his diet during the winter as well as obtain more physical activity during these months.

### 6. EXAMPLE OPERATIONS

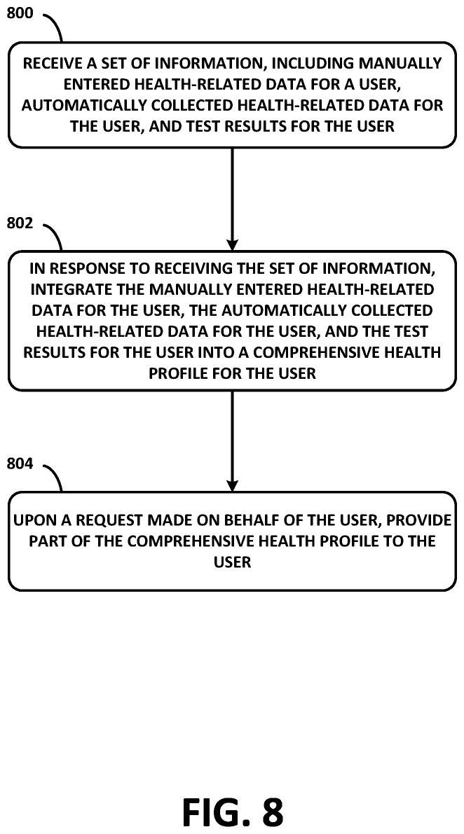

FIG. 8 is a flow chart illustrating an example embodiment. The process illustrated by FIG. 8 may be carried out by a computing device, such as computing device 200, and/or a cluster of computing devices, such as server cluster 304. However, the process can be carried out by other types of devices or device subsystems. For example, the process could be carried out by a portable computer, such as a laptop or a tablet device. Further, the process may be combined with one or more features disclosed in the context of any previous figure.

At block 800, a computing device may receive a set of information including manually entered health-related data for a user, automatically collected health-related data for the user, and test results for the user. In some cases, the computing device may collect this information from various sources.

At block 802, in response to receiving the set of information, the computing device may integrate the manually entered health-related data for the user, the automatically collected health-related data for the user, and the test results for the user into a comprehensive health profile for the user. At block 804, upon a request made on behalf of the user, the computing device may provide part of the comprehensive health profile to the user.

The test results for the user may be based on a first test and a second test. The first test may be performed by a first laboratory and the second test may be performed by a second laboratory. The first laboratory and the second laboratory may be independent from one another.

Receiving the test results for the user may involve receiving a first request for the first test for the user and facilitating shipment of a first kit for the first test to the user. The first test kit may include instructions to use the first kit and to provide a first sample for the first test to the first laboratory. First results of the first test may be received from the first laboratory.

Receiving the test results for the user may also involve receiving a second request for the second test for the user and facilitating shipment of a second kit for the second test to the user. The second test kit may include instructions to use the second kit and to provide a second sample for the second test to the second laboratory. Second results of the second test may be received from the second laboratory.

In some embodiments, integrating the manually entered health-related data for the user, the automatically collected health-related data for the user, and the test results for the user into the comprehensive health profile for the user may involve determining at least one health-related correlation between any two of the manually entered health-related data for the user, the automatically collected health-related data for the user, and the test results for the user. This integration may further involve adding an indication of the health-related correlation to the comprehensive health profile for the user.

Alternatively or additionally, integrating the manually entered health-related data for the user, the automatically collected health-related data for the user, and the test results for the user into the comprehensive health profile for the user may involve determining one or more longitudinal trends regarding one or more of the manually entered health-related data for the user, the automatically collected health-related data for the user, and the test results for the user. This integration may further involve adding an indication of the one or more longitudinal trends to the comprehensive health profile for the user.

The manually entered health-related data for the user may include nutritional information about the user and/or medical questionnaire answers from the user. The automatically collected health-related data for the user may include physical activity information of the user and/or biometric data of the user.

The example embodiment of FIG. 8 may further include receiving authorization from the user to allow a second user to access at least part of the comprehensive health profile for the user, and modifying the comprehensive health profile for the user to allow the second user to access at least part of the comprehensive health profile for the user. Additionally, a request may be received from the second user to access the at least part of the comprehensive health profile for the user, and a representation of the at least part of the comprehensive health profile for the user may be transmitted to the second user.

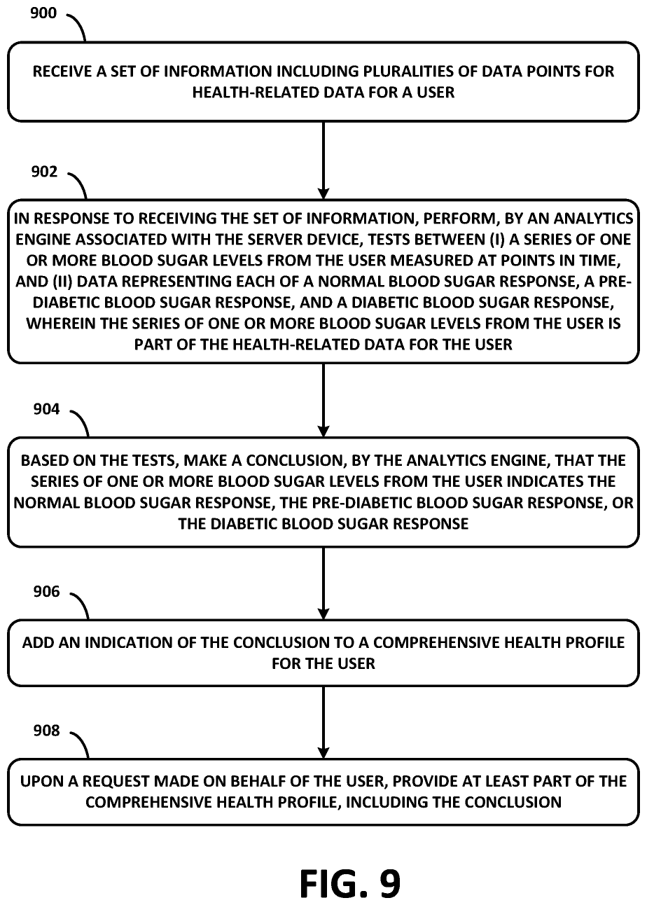

FIG. 9 is another flow chart illustrating another example embodiment. The process illustrated by FIG. 9 may be carried out by a computing device, such as computing device 200, and/or a cluster of computing devices, such as server cluster 304. However, the process can be carried out by other types of devices or device subsystems. For example, the process could be carried out by a portable computer, such as a laptop or a tablet device. Further, the process may be combined with one or more features disclosed in the context of any previous figure.

Block 900 may involve receiving, by a server device, a set of information including pluralities of data points for health-related data for a user.

Block 902 may involve, perhaps in response to receiving the set of information, performing, by an analytics engine associated with the server device, tests between (i) a series of one or more blood sugar levels from the user, and (ii) data representing each of a normal blood sugar response, a pre-diabetic blood sugar response, and a diabetic blood sugar response, wherein the series of one or more blood sugar levels from the user is part of the health-related data for the user.

Block 904 may involve, perhaps based on the tests, making a conclusion, by the analytics engine, that the series of one or more blood sugar levels from the user indicates the normal blood sugar response, the pre-diabetic blood sugar response, or the diabetic blood sugar response.

Block 906 may involve adding, by the server device, an indication of the conclusion to a comprehensive health profile for the user.

Block 908 may involve, upon a request made on behalf of the user, providing, by the server device, at least part of the comprehensive health profile, including the conclusion.

In some embodiments, the series of one or more blood sugar levels are received from a device, and wherein the device is one of: a wearable device, mobile application, mobile tracking device, sensor, textile, or medical device. The device may periodically measure a blood sugar level of the user.

In some embodiments, the series of one or more blood sugar levels are received by way of manual entry from the user.

In some embodiments, the tests are based on regression analysis.

Some embodiments may further involve receiving an indication of when the user has eaten.

In some embodiments, the one or more blood sugar levels from the user are measured at points in time after the user has eaten, wherein the normal blood sugar response, the pre-diabetic blood sugar response, and the diabetic blood sugar response are all responses after eating. In variations, data representing the normal blood sugar response after eating starts at approximately 90 mg/dL immediately after eating, peaks at approximately 110 mg/dL one hour after eating, then returns to approximately 90 mg/dL within two hours after eating. In other variations, the data representing the pre-diabetic blood sugar response after eating starts at approximately 100 mg/dL immediately after eating, peaks at approximately 145 mg/dL between one and two hours after eating, drops to under 80 mg/dL between three and five hours after eating, then returns to approximately 100 mg/dL within seven hours after eating. In other variations, the data representing the diabetic blood sugar response after eating starts at approximately 125 mg/dL immediately after eating, peaks at approximately 215 mg/dL between one and two hours after eating, then returns to approximately 125 mg/dL within seven hours after eating.

In some embodiments, the one or more blood sugar levels from the user are measured at points in time before the user has eaten, wherein the normal blood sugar response, the pre-diabetic blood sugar response, and the diabetic blood sugar response are all responses before eating.

In some embodiments, the one or more blood sugar levels from the user are measured at points in time after the user has awoken, wherein the normal blood sugar response, the pre-diabetic blood sugar response, and the diabetic blood sugar response are all responses after awakening.

In some embodiments, the comprehensive health profile for the user also includes test results based on a first test and a second test, wherein the first test was performed by a first laboratory and the second test was performed by a second laboratory on different parts of a sample from the user, and wherein the first laboratory and the second laboratory are independent from one another.

In some embodiments, the comprehensive health profile for the user also includes one or more longitudinal trends regarding the health-related data for the user.

In some embodiments, the health-related data for the user includes nutritional information about the user.

In some embodiments, the health-related data for the user includes medical questionnaire answers from the user.

In some embodiments, the health-related data for the user includes physical activity information of the user.

Some embodiments may involve: (i) receiving, by the server device, authorization from the user to allow a second user to access part of the comprehensive health profile for the user; (ii) modifying, by the server device, the comprehensive health profile for the user to allow the second user to access the part of the comprehensive health profile for the user; (iii) transmitting, by the server device, a notification to a device associated with the second user, wherein the notification indicates that the second user can access the part of the comprehensive health profile for the user; (iv) receiving, by the server device, a request from the second user to access the part of the comprehensive health profile for the user; and (v) transmitting, by the server device, a representation of the part of the comprehensive health profile for the user to the second user.

### 7. CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The above detailed description describes various features and functions of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages can be executed out of order from that shown or discussed, including substantially concurrent or in reverse order, depending on the functionality involved. Further, more or fewer blocks and/or functions can be used with any of the ladder diagrams, scenarios, and flow charts discussed herein, and these ladder diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical functions or actions in the method or technique. The program code and/or related data can be stored on any type of computer readable medium such as a storage device including a disk, hard drive, or other storage medium.

The computer readable medium can also include non-transitory computer readable media such as computer-readable media that store data for short periods of time like register memory, processor cache, and random access memory (RAM). The computer readable media can also include non-transitory computer readable media that store program code and/or data for longer periods of time. Thus, the computer readable media may include secondary or persistent long term storage, like read only memory (ROM), optical or magnetic disks, compact-disc read only memory (CD-ROM), for example. The computer readable media can also be any other volatile or non-volatile storage systems. A computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

I claim:

1. A method comprising:
receiving, by a server device, a set of information including pluralities of data points for health-related data for a user;
in response to receiving the set of information, performing, by an analytics engine associated with the server device, tests between (i) a series of one or more blood sugar levels from the user, and (ii) data representing each of a normal blood sugar response, a pre-diabetic blood sugar response, and a diabetic blood sugar response, wherein the series of one or more blood sugar levels from the user is part of the health-related data for the user;
based on the tests, making a conclusion, by the analytics engine, that the series of one or more blood sugar levels from the user indicates the normal blood sugar response, the pre-diabetic blood sugar response, or the diabetic blood sugar response;
adding, by the server device, an indication of the conclusion to a comprehensive health profile for the user; and
upon a request made on behalf of the user, providing, by the server device, at least part of the comprehensive health profile, including the conclusion.

2. The method of claim 1, wherein the series of one or more blood sugar levels are received from a device, and wherein the device is one of: a wearable device, mobile application, mobile tracking device, sensor, textile, or medical device.

3. The method of claim 2, wherein the device periodically measures a blood sugar level of the user.

4. The method of claim 1, wherein the series of one or more blood sugar levels are received by way of manual entry from the user.

5. The method of claim 1, wherein the tests are based on regression analysis.

6. The method of claim 1, further comprising:
receiving an indication of when the user has eaten.

7. The method of claim 1, wherein the one or more blood sugar levels from the user are measured at points in time after the user has eaten, wherein the normal blood sugar response, the pre-diabetic blood sugar response, and the diabetic blood sugar response are all responses after eating.

8. The method of claim 7, wherein the data representing the normal blood sugar response after eating starts at approximately 90 mg/dL immediately after eating, peaks at approximately 110 mg/dL one hour after eating, then returns to approximately 90 mg/dL within two hours after eating.

9. The method of claim 7, wherein the data representing the pre-diabetic blood sugar response after eating starts at approximately 100 mg/dL immediately after eating, peaks at approximately 145 mg/dL between one and two hours after eating, drops to under 80 mg/dL between three and five hours after eating, then returns to approximately 100 mg/dL within seven hours after eating.

10. The method of claim 7, wherein the data representing the diabetic blood sugar response after eating starts at approximately 125 mg/dL immediately after eating, peaks at approximately 215 mg/dL between one and two hours after eating, then returns to approximately 125 mg/dL within seven hours after eating.

11. The method of claim 1, wherein the one or more blood sugar levels from the user are measured at points in time before the user has eaten, wherein the normal blood sugar response, the pre-diabetic blood sugar response, and the diabetic blood sugar response are all responses before eating.

12. The method of claim 1, wherein the one or more blood sugar levels from the user are measured at points in time after the user has awoken, wherein the normal blood sugar response, the pre-diabetic blood sugar response, and the diabetic blood sugar response are all responses after awakening.

13. The method of claim 1, wherein the comprehensive health profile for the user also includes test results based on a first test and a second test, wherein the first test was performed by a first laboratory and the second test was performed by a second laboratory on different parts of a sample from the user, and wherein the first laboratory and the second laboratory are independent from one another.

14. The method of claim 1, wherein the comprehensive health profile for the user also includes one or more longitudinal trends regarding the health-related data for the user.

15. The method of claim 1, wherein the health-related data for the user includes nutritional information about the user.

16. The method of claim 1, wherein the health-related data for the user includes medical questionnaire answers from the user.

17. The method of claim 1, wherein the health-related data for the user includes physical activity information of the user.

18. The method of claim 1, further comprising:
receiving, by the server device, authorization from the user to allow a second user to access part of the comprehensive health profile for the user;
modifying, by the server device, the comprehensive health profile for the user to allow the second user to access the part of the comprehensive health profile for the user;
transmitting, by the server device, a notification to a device associated with the second user, wherein the notification indicates that the second user can access the part of the comprehensive health profile for the user;
receiving, by the server device, a request from the second user to access the part of the comprehensive health profile for the user; and
transmitting, by the server device, a representation of the part of the comprehensive health profile for the user to the second user.

19. An article of manufacture including a non-transitory computer-readable medium, having stored thereon program instructions that, upon execution by a server device, cause the server device to perform operations comprising:
receiving a set of information including pluralities of data points for health-related data for a user;
in response to receiving the set of information, performing, by an analytics engine associated with the server device, tests between (i) a series of one or more blood sugar levels from the user measured at points in time, and (ii) data representing each of a normal blood sugar response, a pre-diabetic blood sugar response, and a diabetic blood sugar response, wherein the series of one or more blood sugar levels from the user is part of the health-related data for the user;
based on the tests, making a conclusion, by the analytics engine, that the series of one or more blood sugar levels from the user indicates the normal blood sugar response, the pre-diabetic blood sugar response, or the diabetic blood sugar response;
adding an indication of the conclusion to a comprehensive health profile for the user; and
upon a request made on behalf of the user, providing at least part of the comprehensive health profile, including the conclusion.

20. A server device comprising:
at least one processor;
data storage; and
program instructions, stored in the data storage, that upon execution by the at least one processor cause the server device to perform operations including:
receiving a set of information including pluralities of data points for health-related data for a user;
in response to receiving the set of information, performing, by an analytics engine associated with the server device, tests between (i) a series of one or more blood sugar levels from the user measured at points in time, and (ii) data representing each of a normal blood sugar response, a pre-diabetic blood sugar response, and a diabetic blood sugar response, wherein the series of one or more blood sugar levels from the user is part of the health-related data for the user;
based on the tests, making a conclusion, by the analytics engine, that the series of one or more blood sugar levels from the user indicates the normal blood sugar response, the pre-diabetic blood sugar response, or the diabetic blood sugar response;
adding an indication of the conclusion to a comprehensive health profile for the user; and
upon a request made on behalf of the user, providing at least part of the comprehensive health profile, including the conclusion.
